(19) United States
(12) Patent Application Publication
(10) Pub. No.: US 2005/0177047 A1
(43) Pub. Date: Aug. 11, 2005

(54) Device for, and a method of, transcutaneous pressure waveform sensing of an artery and a related target apparatus

(76) Inventors: Peter Chris Harpas, Chipping Norton (AU); Yervant Chijian, Marsfield (AU)

Correspondence Address: BRINKS HOFER GILSON & LIONE, P.O. BOX 10395, CHICAGO, IL, 60610, US

(21) Appl. No.: 10/510,164

(22) PCT Filed: Mar. 28, 2003
(86) PCT No.: PCT/AU03/00386

(30) Foreign Application Priority Data
Apr. 2, 2002 (AU) PS1472

(51) Int. Cl. A61B 5/02

(52) U.S. Cl. 600/485; 600/490

(57) ABSTRACT

A device (20) for transcutaneous pressure waveform sensing of an artery. The device (20) having, in use, an application direction (43) towards the skin (46) of a user. The device (20) includes a pressure sensing head (37) having a distal end (36) and at least one skin depressing means (38) extending at least partially around the head (37) and having a distal surface (40). The pressure sensing head distal end (36) and skin depressing means distal surface(s) (40) are sized such that the pressure sensing head distal end (36) is spaced apart, in the application direction, from the skin depressing means distal surface(s) (40). A method of transcutaneous pressure waveform sensing and a target apparatus (100) are also disclosed.

### FIELD OF THE INVENTION

The present invention relates to a device for, and a method of transcutaneous pressure waveform sensing of an artery and a related target apparatus. Transcutaneous pressure waveform sensing is also sometimes referred to as arterial tonometry or transcutaneous blood pressure pulse waveform sensing.

### BACKGROUND OF THE INVENTION

Transcutaneous pressure waveform sensing involves applying a tonometer (ie. a pressure sensor) to the skin of a patient over a superficial artery, such as the radial artery. The tonometer is applied with enough downward force to applanate (ie. partially flatten) the underlying artery. When applanated, the pressure pulses are no longer taken up circumferentially in the artery wall, but instead are transferred through the artery wall and surrounding tissue in a radial direction of the applanating force to the interface between the skin and the small area of the tonometer above the artery at which point the intra-arterial pressure waveform can now be faithfully recorded. The varying pulsating force applied to the tonometer by the transmitted intra-arterial pressure pulse waveform is continuously converted into an electrical signal by a pressure transducer and plotted on a screen display which allows a clinician to examine the intra-arterial pressure waveform of the patient. Analysing the pressure pulse waveform using known appropriate software can provide diagnostic information to clinicians in relation to areas such as: stiffness of arteries; susceptibility to myocardial ischaemia; risk stratification of marginal hypertensives; and cardiovascular risk of type II diabetic patients.

The Colin Medical CBM-7000 is a known transcutaneous pressure sensing device that consists of a pressure pulse waveform detecting apparatus which is strapped around the wrist, a sphygmomanometer cuff which is wrapped around the arm, and a screen display. The pressure pulse wave detecting apparatus has a chamber housing a semiconductor pressure sensor array. The pressure sensor array is positioned above the radial artery, and using hydraulic pressure, is pushed downwards to suitably flatten the artery. The amount of pressure applied to the sensor array is dependent on the output signal recorded by these sensors. A central processing unit (CPU) examines the output signals and gives a signal to a pressure valve to either reduce or increase the downward pressure on the pressure sensor array. In other words, the CPU chooses the correct amount of tonometer downward pressure needed to applanate the artery sufficiently to obtain a strong arterial pressure waveform signal. A similar technique is used to correctly position the transducers just above the radial artery. More particularly, if a strong pressure waveform signal does not appear on any of the array of sensors, a servomotor will move the sensor array laterally until one of the sensors has a suitably strong signal, again with the CPU comparing all output signals from the transducers. This is a time consuming process. At the end of this operation, the output signal will resemble the intra arterial blood pressure waveform, but in an analogue voltage signal form. A sphygmomanometer cuff is then used to determine brachial artery maximum (“systolic”) and minimum (“diastolic”) pressures and the transducer pulse waveform recorded by the radial artery tonometer is calibrated according to these maximum/minimum values. The output monitor then displays a continuous and calibrated radial blood pressure waveforms. A disadvantage of this device is that it is complicated and time consuming to set up for a measurement. Another disadvantage is that the device is expensive initially, and has high in-service costs due to the electromechanical complexity of the pressure sensor module that is attached to the wrist.

The Millar Instruments SPT-301B Tonometer is another known transcutaneous pressure sensing device that is a hand-held pen-like device that contains a strain gauge on the end that converts a mechanical force into an electrical signal. For the tonometer to sense the arterial pressure, a downward force on the tonometer over the underlying artery is manually applied by the user to applanate the artery. With this downward pressure, the pressure transducer on the end of the tonometer in contact with the skin will begin to sense the changing force resulting from the intra-arterial blood pressure pulsations. This tonometer is presently internationally accepted as the most accurate sensor for making transcutaneous arterial pressure waveform recordings. The size of the pressure sensing transducer in the Millar tonometer is very small and as a result it has the disadvantage that it has to be precisely positioned above the radial artery to achieve accurate results, which is difficult. Another disadvantage of the Miller tonometer is that, because it is pencil-shaped, it flattens only a small area when the sensing end is pushed over the artery, and this can lead to the artery moving laterally out from under the sensor into the uncompressed area and so giving no arterial pressure waveform signal in the sensor.

The Hypertension Diagnostics Inc CD-2000 is another known transcutaneous arterial pressure sensing device that includes a fixed pressure sensor that is strapped over the radial artery. The operator rotates a screw-threaded wheel to push the pressure sensor down over the artery and applanate the artery until a strong arterial pressure is recorded. The head of the device in contact with the skin is 1˜1.5 cms in diameter. Disadvantages of the CD-2000 device are it is complicated and time consuming to set up for a measurement and it is expensive. Further, it has not been shown that this tonometer is able to faithfully reproduce the intra-arterial waveform.

It is an object of the present invention to substantially overcome or at least ameliorate one or more of the deficiencies of the prior art devices discussed above.

### SUMMARY OF THE INVENTION

Accordingly, in a first aspect, the present invention provides a device for transcutaneous pressure waveform sensing of an artery, the device having, in use, an application direction towards the skin of a user in the direction of an underlying artery and including:
a pressure sensing head having a distal end; and
at least one skin depressing means substantially adjacent the pressure sensing head and having a distal surface, wherein the pressure sensing head distal end and skin depressing means distal surface(s) are sized such that the pressure sensing head distal end is spaced apart, in the application direction, from the skin depressing means distal surface(s).

In one form, the pressure sensing head distal end protrudes from the skin depressing means distal surface(s) in the application direction.

In another form, the skin depressing means distal surface(s) protrudes from the pressure sensing head distal end in the application direction.

In an embodiment, the distance between the skin depressing means distal surface and the pressure sensing head distal end is fixed. The distance between the skin depressing means distal surface(s) and the pressure sensing head distal end is preferably approximately 1.5 mm to 2.0 mm, for the radial artery.

In another embodiment, the distance between the skin depressing means distal surface(s) and the pressure sensing head distal end is variable. The device preferably includes a handle and the distance between the skin depressing means distal surface(s) and the handle varies relative to the application pressure applied to the user's skin and the distance between the pressure sensing head distal end and the handle is fixed. In this form, the skin depressing means is/are preferably formed from a compressible material. Alternatively, the device includes a handle and the distance between the pressure sensing head distal end and the handle varies relative to the application pressure applied to the user's skin and distance between the skin depressing means distal surface(s) and the handle is fixed. In this form, the device preferably includes a compression spring arrangement between the pressure sensing head and the handle.

In one arrangement, the device preferably has a single skin depressing means. In this arrangement, the skin depressing means has a substantially annular distal surface.

In another arrangement, the device has a single skin depressing means positioned, in use, on one side of the artery.

In a further arrangement, the device has a pair skin depressing means positioned, in use, either side of the artery. In a preferred from of this arrangement, the skin depressing means distal surface(s) are each hemispherical. In another form of this arrangement, the skin depressing means distal surface(s) are each oriented substantially normally to the longitudinal direction of the artery.

In a second aspect, the present invention provides a method of transcutaneous pressure waveform sensing of an artery, the method including the steps of:
flattening and depressing at least some of the skin around the artery and displacing same, in an application direction towards the artery, to a first depth; and
flattening and depressing the skin over the artery and displacing same, in the application direction, to a second depth that differs than the first depth.

In one form, the first depth is greater than the second depth.

In another form, the second depth is greater than the first depth.

In one embodiment, the distance between the first depth and the second depth is fixed. The distance between the first depth and the second depth is preferably approximately 1.5 mm to 2.0 mm.

In another embodiment, the distance between the first depth and the second depth is variable.

In one arrangement, the method includes flattening, depressing and displacing a circular region of the skin over the artery. In this arrangement, the method also preferably includes flattening, depressing and displacing an annular region of the skin around the artery.

In another arrangement, the method includes flattening, depressing and displacing the skin around the artery on one side of the artery.

In a further arrangement, the method includes flattening, depressing and displacing the skin around the artery on both sides of the artery. In a preferred form of this arrangement, the method preferably includes flattening, depressing and displacing a pair of hemispherical regions of the skin around the artery, the regions being either side of the artery. In another form of this arrangement, the method preferably includes flattening, depressing and displacing a pair of regions of the skin around the artery, the regions being either side of the artery and oriented substantially normally to the longitudinal direction of the artery.

In a third aspect, the present invention provides a target apparatus for use with a device for transcutaneous waveform pressure sensing of an artery, the device having an end pressure sensing head protruding therefrom, the apparatus including:
an skin adhesive pad;
a target marking on the pad; and
skin compression means at least partially around the target marking,
wherein, during use, the device is applied to the pad with the device pressure sensing head over the target marking and the device end over the skin compression means whereby, when the device is depressed into the skin during use, the skin beneath the target marking and the skin compression means are displaced to differing depths.

The target marking is preferably substantially circular.

The skin compression means are preferably a pair of substantially rectangular pads either side of the target marking.

In a fourth aspect, the present invention provides a device for transcutaneous pressure waveform sensing, the device including:
a pressure sensing head having a distal end; and
a collar extending at least partially around the head and having a distal surface, wherein the pressure sensing head distal end and collar are sized such that the pressure sensing head protrudes from the collar distal surface.

In use, when applied to a patient's skin over an underlying artery, at least some of the skin around the artery is flattened and depressed by the collar to a first depth and the skin over the artery is flattened and depressed by the pressure sensing head distal end to a second depth greater than the first depth.

In a fifth aspect, the present invention provides a method of transcutaneous pressure waveform sensing, the method including the steps of:
flattening and depressing at least some of the skin around an underlying artery and displacing same to a first depth; and
flattening and depressing the skin over the underlying artery and displacing same to a second depth greater than the first depth.

The skin around the artery is preferably compressed by the collar either side of the longitudinal direction of the artery (ie. the lateral sides). The width of the skin area flattened (and thus the width of the collar) either side of the artery, in a direction normal to the longitudinal direction of the artery, is dependent upon the different physiology surrounding the specific artery (eg, radial, carotid, femoral). For the radial artery, it is desirable to compress about 3 times the unflattened width of the artery in each direction lateral to the direction of the artery.

For the radial artery the difference between the first and second depth is preferably approximately 1.5-2.0 mm.

For use with the radial artery, the collar is configured to be symmetrical in relation to the longitudinal direction of the artery and non-symmetrical in relation to a direction normal to the longitudinal direction of the artery, with the length of the collar from the pressure sensing head towards the patient's wrist being smaller than the length of the collar from the pressure sensing head towards the patient's elbow.

For use with the carotid artery, the collar is configured to be non-symmetrical in relation to the longitudinal direction of the artery and non-symmetrical in relation to a direction normal to the longitudinal direction of the artery, with the length of the collar from the pressure sensing head towards the front of the patient's throat being smaller than the length of the collar from the pressure sensing head towards the patient's neck and the length of the collar from the pressure sensing head towards the patient's shoulder being smaller than the length of the collar from the pressure sensing head towards the patient's head.

The article entitled “Arterial Tonometry: Review and Analysis” by Gary M. et al (J. Biomechanics Vol. 16. No. 2. pp. 141-152, 1983) sets out the optimal width of the pressure sensing head relative to uncompressed artery diameter. The preferred diameter of the pressure sensing head is less than or equal to the width of the flattened section of the underlying artery. However, for maximum flexibility in positioning the sensor, it is desirable to have the pressure sensing head as wide as possible (witness the limitations of the Millar sensor).

In one preferred form for the radial artery, the pressure sensing head is circular in cross section and the collar is annular in cross section, and the diameter of the pressure sensing head is approximately 3.5 mm.

### BRIEF DESCRIPTION OF THE DRAWINGS

A preferred embodiment of the invention will now be described, by way of example only, with reference to the accompanying drawings in which:

FIG. 1 is a cross-sectional side view of a first embodiment of a device for transcutaneous pressure waveform sensing according to the invention;

FIG. 2 is a top view of a collar used with the device shown in FIG. 1;

FIG. 3 is a bottom view of the collar shown FIG. 2;

FIG. 4 is a side view of the collar shown in FIG. 2;

FIG. 5 is a cross-sectional side view of the collar shown in FIG. 2;

FIG. 6 is a cross-sectional side view of the device shown in FIG. 1 without the collar;

FIG. 7 is a front view of the device shown in FIG. 6;

FIG. 8 is a top view of the device shown in FIG. 6;

FIG. 9 is a side view of the device shown in FIG. 6;

FIG. 10 is a bottom view of the device shown in FIG. 6;

FIG. 11 is a side view of the device shown in FIG. 1 over a patient's skin, prior to use;

FIG. 12 is a side view of the device shown in FIG. 1, during use.

FIG. 13 is an inverted side view of a second embodiment of a device for a transcutaneous pressure waveform sensing according to the invention;

FIG. 14 is a bottom view of the device shown in FIG. 13;

FIG. 15 is an inverted front view of the device shown in FIG. 13;

FIG. 16 is an inverted side view of a third embodiment of a device for transcutaneous pressure waveform sensing according to the invention;

FIG. 17 is a bottom view of the device shown in FIG. 16;

FIG. 18 is an inverted front view of the device shown in FIG. 16;

FIG. 19 is an exaggerated cross sectional side view of the device shown in FIG. 16 during use;

FIG. 20 is an inverted side view of a fourth embodiment of a device for a transcutaneous pressure waveform sensing according to the invention;

FIG. 21 is a bottom view of the device shown in FIG. 20;

FIG. 22 is an inverted front view of the device shown in FIG. 20;

FIG. 23 is a side view of a first embodiment of a target apparatus according to the invention;

FIG. 24 is a top view of the apparatus shown in FIG. 23; and

FIG. 25 is a side view of the apparatus shown in FIG. 23 with an adjacent transcutaneous waveform pressure sensing device.

### DESCRIPTION OF THE PREFERRED EMBODIMENT

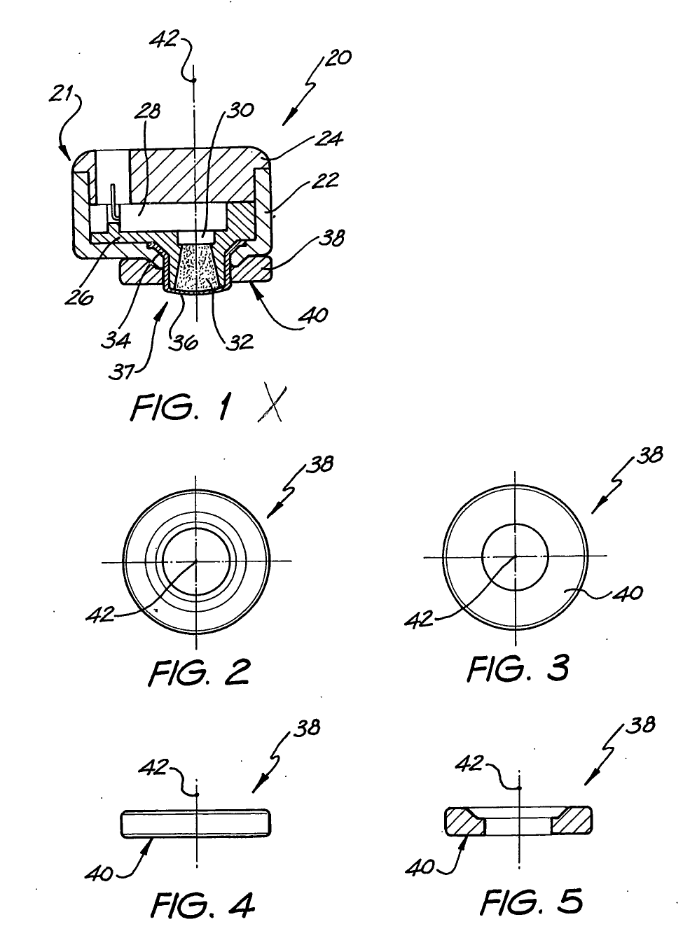

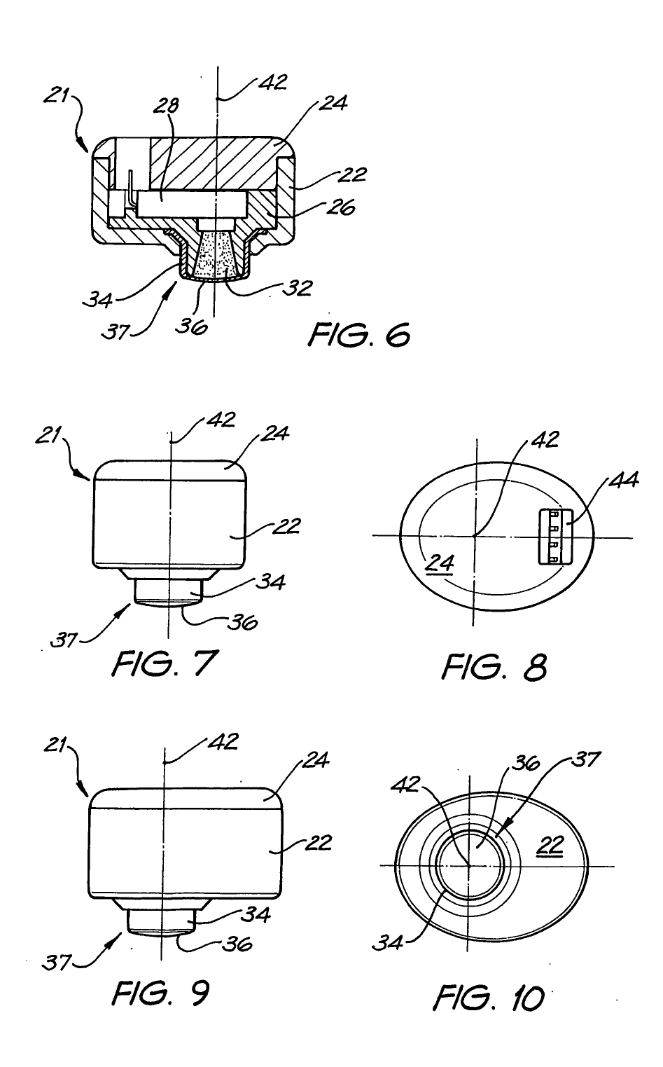

Referring to the FIGS. 1 to 12, there is shown a device 20 for transcutaneous pressure waveform sensing according to a first embodiment of the invention. The device 20 is comprised of a housing 21 formed from a base 22 and top 24, which are both formed from poly vinyl chloride (PVC).

An internal chassis 26 within the base 22 supports a pressure transducer 28, in the form of a Pressure Sensor model No. MPX2300DT1, produced by the Motorola Company. The chassis 26 also includes an opening 30 which allows the sensor 28 to communicate pressure changes brought about in a pocket of gel 32, preferably dielectric silicone gel—grade 537 produced by the Dow Corning Company. The gel 32 is retained in the chassis 26 by a silicone boot 34, preferably of shore hardness 55.

The exterior surface of the boot 34 that is denoted 36 represents the distal end of an overall “pressure sensing head” 37 formed by components 28, 30, 32 and 34.

The device 20 also include a “skin depression means” in the form of an annular PVC collar 38 around the head 37. The collar 38 has a distal surface denoted 40, an external diameter of 13.6 mm and a thickness in the direction of axis 40 of 2.5 mm. The internal diameter of the collar 38 is approximately 6 mm and is a snug fit around the cylindrical side wall of the boot 34. The collar 38 is an interference locating fit over the boot 34.

It is important to note that the distal end 36 of the pressure sensing head 37 protrudes in the direction of axis 42 from the distal surface 40 of the collar 38. It is also important to note that the axis is substantially parallel to what is termed the “application direction”, being the general direction (as represented by arrow 43) the device 20 is applied, in use, towards the skin and the underlying artery.

As best shown in FIG. 8, the device 20 also include an electrical connection 44 which allow electrical signals generated by the HVPS 28 to be communicated to appropriate computer equipment and processing software, as is well known in the art and which will not be described further.

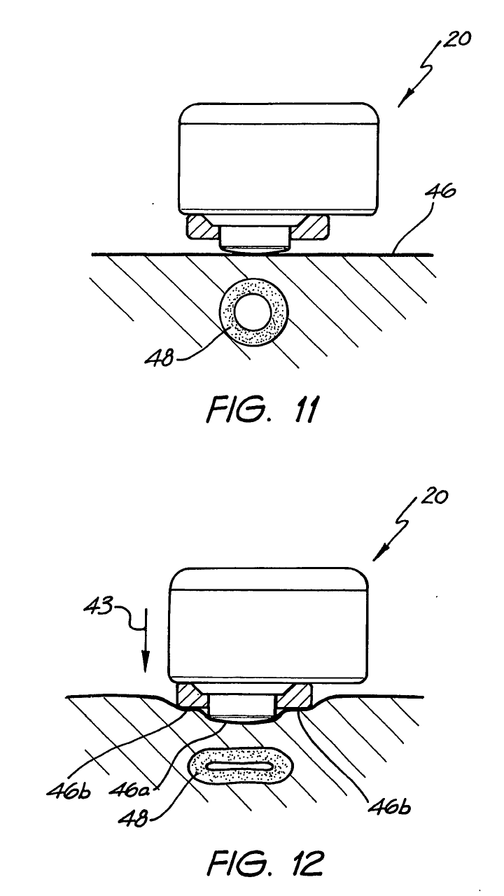

The operation of the device 20 will now be described with particular reference to FIGS. 11 and 12. FIG. 11 shows the device 20 positioned over the skin 46 above the radial artery 48 of a human. FIG. 12 shows the device 20 after depression into the skin 46 in the application direction to cause flattening of the artery 48. As the distal end 36 of the pressure sensing head 37 protrudes from the distal surface 40 of the collar 38. This results in the skin 46a adjacent the pressure sensing head 37 being displaced about 2 mm more than the skin 46b adjacent the collar 38, which stretches the tissue beneath the skin 46b (ie. the tissue beneath the collar 38).

The correct amount of depression of the device 20 into the skin 46 is determined by the clinician manipulating the device 20 whilst watching a graphical representation of the intra-arterial pressure profile of the patient on a display screen (not shown). If the device 20 has not been depressed into the skin 46 far enough, a low amplitude and noisy pulse waveform signal will be evident. If the device 20 has been depressed too far into the skin 46 any pulse waveform signal will be lost due to the artery 46 being excessively flattened into occlusion. The clinician can determine when the device 20 has been optimally depressed into the skin 46 when a large amplitude pulse waveform signal is evident on the display screen.

The clinician can also choose collars of different thicknesses to adjust the relative amounts of depression, depending on the type and location of artery and the physical characteristics of the patient (eg. age, muscle tone).

The device 20 provides a superior quality signal than prior art devices because it compresses the soft tissue on either side of the artery 48 which prevents the artery 48 from moving laterally away from the distal end 36 of pressure sensing head during applanation.

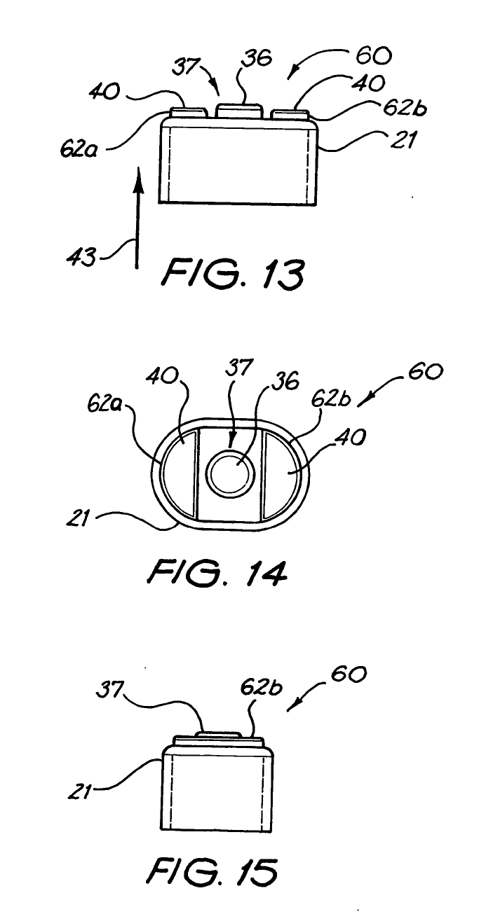

Referring to FIGS. 13 to 15 there is shown a device 60 for transcutaneous pressure waveform sensing according to a second embodiment of the invention. The device is similar to the first embodiment shown in FIGS. 1 to 12 and like reference numerals will be used to indicate like features. The main difference between the devices 20 and 60 is that the device 60 has a pair of skin depression means in the form of solid foam pads 62a and 62b. During use, the device 60 is oriented such that the pads 62a and 62b lie either side of the artery being measured. Similar to the first embodiment, the device 60 provides a superior quality signal than prior art devices because it compresses the soft tissue on either side of the artery, which thereby prevents the artery from moving laterally away from the distal end 36 of the pressure sensing head 37 during applanation.

Similar to the first embodiment, the clinician can also choose pads of different thicknesses to adjust the relative amounts of depression.

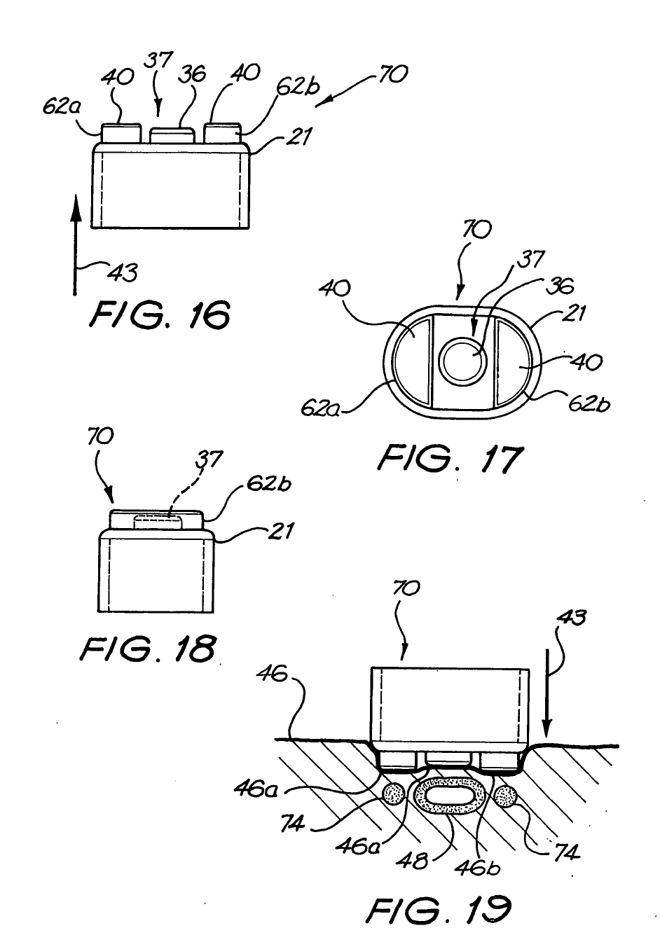

Referring to FIGS. 16 to 19 there is shown device 70 for transcutaneous pressure waveform sensing according to a third embodiment of the invention. The device 70 is similar to the device 60 and like reference numerals will be used to denote like features. The main difference between the devices 60 and 70 is that, in the device 70, the distal surfaces 40 of the two pads 62a and 62b are displaced further away from the housing 21 of the device 70 than the distal end 36 of the pressure sensing head 37.

During use, the device 70 depresses and displaces the skin 46a above the artery 48 and the skin 46b either side of the artery 48 in the manner indicated by the (exaggerated) representation shown in FIG. 19. In addition to the advantages provided by the earlier embodiments, the device 70 finds particular application in sensing an artery 48 between tendons or ligaments 74 as the pads 62a and 62b result in additional downwards displacement of such tendons or ligaments to allow better access to the artery 48 for applanation.

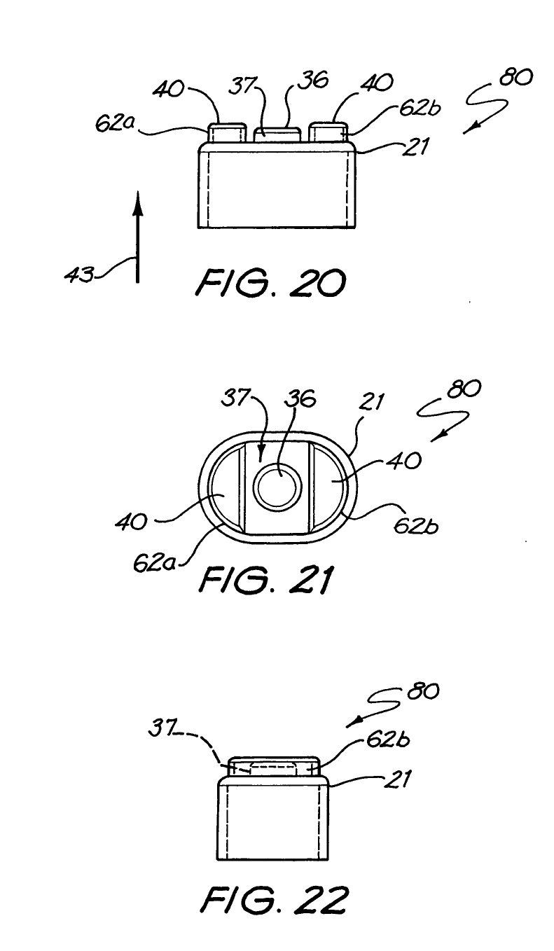

Referring to FIGS. 20 to 22 there is shown a device 80 for transcutaneous pressure waveform sensing according to a fourth embodiment of the invention. The device 80 is similar to the device 70 and like reference numerals will be used to denote like features. The main difference between the device 80 and 70 is that the pads 62a and 62b in the device 80 are formed from hollow foam which are compressible in response to the pressure applied in the application direction towards the skin 46. This allows the clinician to vary the amount of relative displacement between the region of skin 46a above the artery 48 and the regions of skin 46b either side of the artery 48.

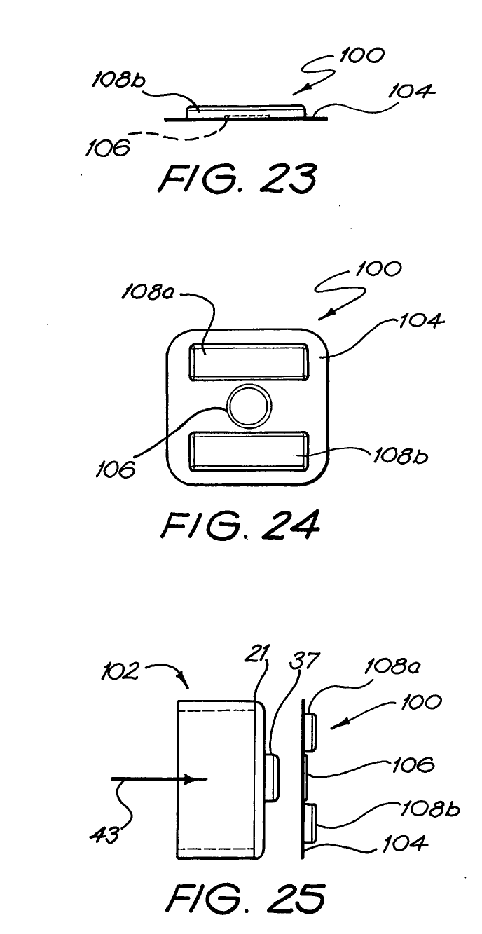

FIGS. 23 to 25 show a target apparatus 100 according to a first embodiment of the invention. The apparatus 100 is used with a device 102 for transcutaneous waveform pressure sensing. The device 102 is similar to the devices 60, 70 and 80 accept it does not have the pads 62a and 62b and instead only has the pressure sensing head 37 solely protruding from the housing 21. The target apparatus 100 has a skin adhesive pad 104 a target marking 106 on the pad 104 and skin depression means, in the form of foam pads 108a and 108b.

In use, the target apparatus 100 is applied to the skin centered above the artery to be measured and with the pads 108a and 108b oriented either side of the artery. The target marking 106 represents where the pressure sensing head 37 of the device 100 should be positioned during use. When the pressure sensing device 102 is depressed into the skin of the user above the target apparatus 100, the skin 46a beneath the target marking (and thus beneath the pressure sensing head 37) is displaced to a different depth to the skin beneath the pads 108a and 108b. This results in the skin being displaced in a similar manner to that is shown in FIG. 12.

The preferred embodiments of devices and methods of use according to the invention are easier to operate and provide superior quality signals than prior art devices/methods. Generally speaking, this is because they ‘condition’ the skin and underlying physiology surrounding the artery in order to better locate and sense the pressure waveform in the underlying artery of interest. They also allow the use of an optimally-sized pressure sensing head, which is able to pick up an accurate signal from the applanated artery, that, due to the skin depressing means, does not need to be as accurately positioned to the centre of the underlying artery as prior art devices (eg, the Millar tonometer). The latter is firstly because the reduced depression of the skin at least on the (lateral) sides of the artery assists in maintaining the preferred symmetrical position between the pressure sensing head and the artery by reducing movement of the artery laterally away from the pressure sensing head during applanation. Such movement is a particular problem when attempting to monitor the pressure of an artery with nearby collateral anatomy such as tendons and muscle etc. The partial depression of the skin lateral to the artery provides a stabilising pressure which maintains the artery in the optimal position relative to the pressure sensing head. Secondly, the width of the pressure sensing head is optimal for the underlying artery according to the Drzewiecki research, which is not the case for the prior art devices.

The preferred size and shape of the pressure sensing head and collar, and the amount by which the distal end of the head protrudes, are determined by trial and error and are dependent on the type, size and location of the superficial artery which is to be measured, and the anatomy surrounding the artery.

Although the invention has been described with reference to a preferred embodiment, it will be appreciated by those skilled in the art that the invention may be embodied in many other forms. For example:
(a) the sensor may be hand-held or be hands-free with some wrist attachment mechanism;
(b) there may be some mechanical mechanism for applying the downward applanation pressure;
(c) the sensor may have fixed or replaceable collars;
(d) different collars may be chosen for the same artery site depending upon the depth of the artery below the skin in a particular patient;
(e) there may be integrated into the sensor construction a mechanism for moving an adjustable-depth collar up and down to vary the height difference between the sensor and collar to an optimal height during the study;
(f) the pressure sensing area may be other than circular; and
(g) the pressure sensor may operate in a sensing method other than described here for example, pressure may be sensed across the sensing area by piezoelectric sensor, by a strain-gauge pressure sensors, by a fibre optic pressure sensors, etc.

1. A device for transcutaneous pressure waveform sensing of an artery, the device having, in use, an application direction towards the skin of a user in the direction of an underlying artery and including:
a pressure sensing head having a distal end; and
at least one skin depressing means substantially adjacent the pressure sensing head and having a distal surface, wherein the pressure sensing head distal end and skin depressing means distal surface(s) are sized such that the pressure sensing head distal end is spaced apart, in the application direction, from the skin depressing means distal surface(s).

2. The device as claimed in claim 1, wherein the pressure sensing head distal end protrudes from the skin depressing means distal surface(s) in the application direction.

3. The device as claimed in claim 1, wherein the skin depressing means distal surface(s) protrudes from the pressure sensing head distal end in the application direction.

4. The device as claimed in claim 1, wherein the distance between the skin depressing means distal surface and the pressure sensing head distal end is fixed.

5. The device as claimed in claim 4, wherein distance between the skin depressing means distal surface(s) and the pressure sensing head distal end is approximately 1.5 mm to 2.0 mm.

6. The device as claimed in claim 1, wherein distance between the skin depressing means distal surface(s) and the pressure sensing head distal end is variable.

7. The device as claimed in claim 6, wherein the device includes a handle and the distance between the skin depressing means distal surface(s) and the handle varies relative to the application pressure applied to the user's skin and the distance between the pressure sensing head distal end and the handle is fixed.

8. The device as claimed in claim 6, wherein the skin depressing means is/are formed from a compressible material.

9. The device as claimed in claim 6, wherein the device includes a handle and the distance between the pressure sensing head distal end and the handle varies relative to the application pressure applied to the user's skin and distance between the skin depressing means distal surface(s) and the handle is fixed.

10. The device as claimed in claim 9, wherein the device includes a compression spring arrangement between the pressure sensing head and the handle.

11. The device as claimed in claim 1, wherein the device has a single skin depressing means.

12. The device as claimed in claim 11, wherein the skin depressing means has a substantially annular distal surface.

13. The device as claimed in claim 1, wherein the device has a single skin depressing means positioned, in use, on one side of the artery.

14. The device as claimed in claim 1, wherein the device has a pair skin depressing means positioned, in use, either side of the artery.

15. The device as claimed in claim 14, wherein the skin depressing means distal surface(s) are each hemispherical.

16. The device as claimed in claim 14, wherein the skin depressing means distal surface(s) are each oriented substantially normally to the longitudinal direction of the artery.

17. A method of transcutaneous pressure waveform sensing of an artery, the method including the steps of:
flattening and depressing at least some of the skin around the and displacing same, in an application direction towards the artery, to a first depth; and
flattening and depressing the skin over the artery and displacing same, in the application direction, to a second depth that differs than the first depth.

18. The method as claimed in claim 17, wherein the first depth is greater than the second depth.

19. The method as claimed in claim 17, wherein the second depth is greater than the first depth.

20. The method as claimed in claim 17, wherein the distance between the first depth and the second depth is fixed.

21. The method as claimed in claim 21, wherein the distance between the first depth and the second depth is approximately 1.5 mm to 2.0 mm.

22. The method as claimed in claim 17, wherein the distance between the first depth and the second depth is variable.

23. The method as claimed in claim 17, including flattening, depressing and displacing a circular region of the skin over the artery.

24. The method as claimed in claim 17, including flattening, depressing and displacing an annular region of the skin around the artery.

25. The method as claimed in claim 17, including flattening, depressing and displacing the skin around the artery on one side of the artery.

26. The method as claimed in claim 17, including flattening, depressing and displacing the skin around the artery on both sides of the artery.

27. The method as in claimed claim 26, including flattening, depressing and displacing a pair of hemispherical regions of the skin around the artery, the regions being either side of the artery.

28. The method as claimed in claim 26, including flattening, depressing and displacing a pair of regions of the skin around the artery, the regions being either side of the artery and oriented substantially normally to the longitudinal direction of the artery.

29. A target apparatus for use with a device for transcutaneous waveform pressure sensing of an artery, the device having an end with a pressure sensing head protruding therefrom, the apparatus including:
an skin adhesive pad;
a target marking on the pad; and
skin compression means at least partially around the target marking, wherein, during use, the device is applied to the pad with the device pressure sensing head over the target marking and the device end over the skin compression means whereby, when the device is depressed into the skin during use, the skin beneath the target marking and the skin compression means are displaced to differing depths.

30. The apparatus of claim 29, wherein the target marking is substantially circular.

31. The apparatus of claim 30, wherein the skin compression means are a pair of substantially rectangular pads either side of the target marking.
